(19) United States
(12) Plant Patent Application Publication
(10) Pub. No.: US 2014/0373229 P1
(43) Pub. Date: Dec. 18, 2014

(54) Clematis plant named 'Evipo043'

(50) ;

(71) Applicants: Mogens Nyegaard Olesen, Fredensborg (DK); Raymond Evison, St. Sampsons (GB)

(72) Inventors: Mogens Nyegaard Olesen, Fredensborg (DK); Raymond Evison, St. Sampsons (GB)

(21) Appl. No.: 13/986,919

(22) Filed: Jun. 14, 2013

(51) Int. Cl. A01H 5/00

(52) U.S. Cl. PLT/228

(57) ABSTRACT

A new Clematis plant with a compact growth habit, profuse, Red-Purple flowers, and continuous summer flowering. The variety successfully propagates from softwood cuttings and is suitable for cultivation commercial nursery culture. This new and distinct variety has shown to be uniform and stable in the resulting generations from asexual propagation from vegetative cuttings.

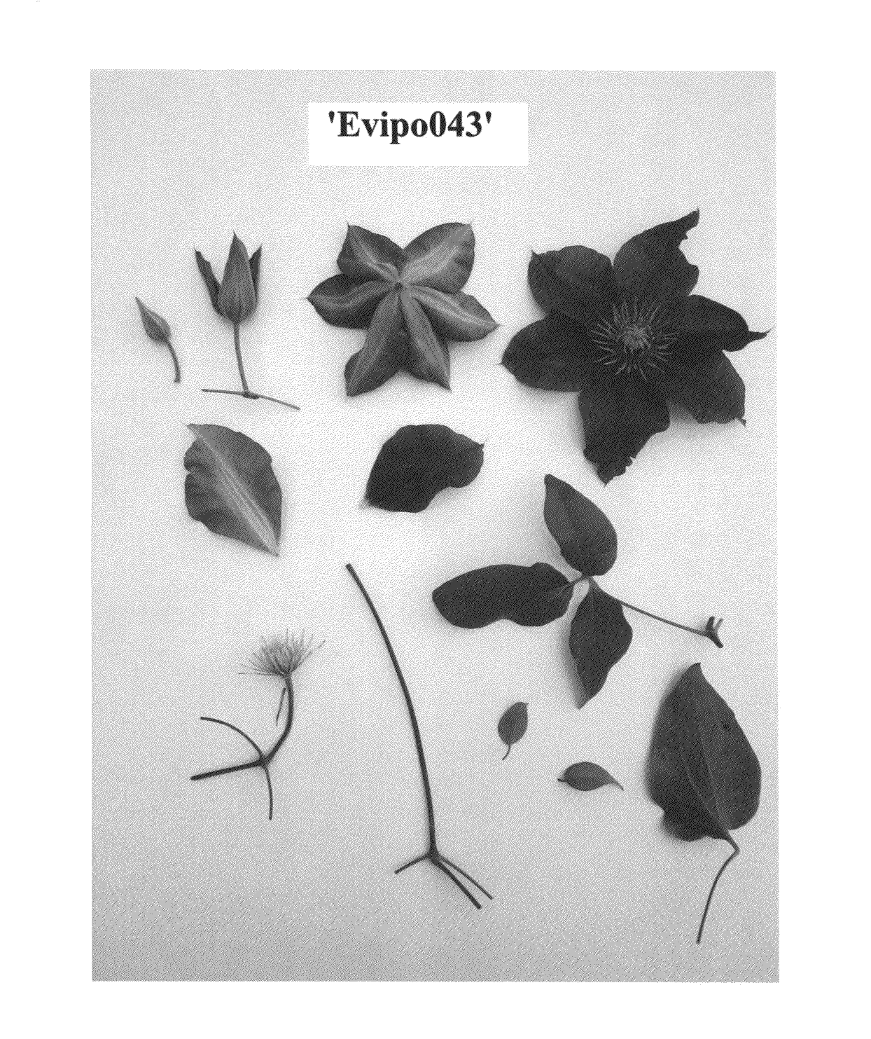

### BOTANICAL CLASSIFICATION

Genus: Clematis

Species: viticella

### VARIETY DENOMINATION

‘Evipo043’

### SUMMARY OF THE CLAIMED PLANT

The present invention constitutes a new and distinct variety of Clematis plant which originated from a controlled crossing between the female seed parent, an un-named seedling, and the male pollen parent, an un-named seedling

The two parents were crossed during the summer of 1996 and the resulting seeds were planted the following winter in a controlled environment in Guernsey, Channel Islands. The new variety named ‘Evipo043’ originated as a single seedling from the stated cross.

The new clematis plant may be distinguished from its female seed parent and male pollen parent primarily by flower color and growth habit.

The objective of the hybridization of this clematis plant was to create a new and distinct variety for nursery culture with unique qualities such as:

1. Uniform and abundant Red-Purple flowers;

2. Vigorous and compact growth, making the variety suitable for container culture;

3. Flowering from new and old growth; and

4. Improved disease resistance.

This combination of qualities was lacking in clematis plants that were in commercial cultivation and the qualities have been substantially achieved in the new variety.

‘Evipo043’ was selected by Mogens N. Olesen and Raymond J. Evison in their clematis development program in the Channel Islands, United Kingdom in 1997 . Asexual reproduction of ‘Evipo043’ by means of vegetative cuttings and traditional layering was first performed by Mogens N. Olesen and Raymond J. Evison in the nursery during the summer of 1997. This initial and subsequent asexual propagations have demonstrated that the characteristics of ‘Evipo043’ are true to type and are transmitted from one generation to the next.

### BRIEF DESCRIPTION OF THE DRAWING

The accompanying color illustration show as true as is reasonably possible to obtain in color photographs of this type the typical characteristics of the buds, flowers, leaves, and stems, of ‘Evipo043’. Specifically illustrated in the drawing are flowers at various stages of development, flower in parts, leaves, and stems.

### DETAILED DESCRIPTION OF THE VARIETY

The following is a detailed description of ‘Evipo043’, as observed in its growth in Yamhill County, Oreg. Observed plants were cultivated for a period of 24 months in 2 gallon containers. Certain phenotypical characteristics of the variety may vary under different environmental, cultural, agronomic, seasonal, and climatic conditions. Color references are made using The Royal Horticultural Society (London, England) Colour Chart, 2001, except where common terms of color are used.

For a comparison, two key characteristics of the clematis variety ‘Evipo020’ described and illustrated in U.S. Plant Pat. No. 16,012, issued Sep. 27, 2005, are compared to ‘Evipo043’ in Chart 1.

CHART 1

| | ‘Evipo043’ | ‘Evipo020’ |
|---|---|---|
| Flower diameter | 120 mm | 80-90 mm |
| Coloration of | Red-Purple | Purple |
| upper tepal surface | Group 71A | Group 79C |

Flower and flower bud:
   Blooming habit.—Continuous. The natural flowering period is generally from May to October.
   Flower bud.—Size: Normally 22 mm in length. Bud diameter is 10 mm. Bud form: Short, broad based, pointed, ellipse. Bud color: At ¼ opening Yellow-Green Group 145A with intonations of Purple Group 76A.
   Peduncle.—Surface Texture: Smooth and pubescent. Length: On average 30 mm in length by 2 mm wide. Color: Yellow-Green Group 145A. Strength: Moderately strong.
   Flower arrangement.—Location on vine: New and old growth. Borne: Singly.
   Flower bloom.—Size: On average, flowers are 120 mm in diameter and 25 mm in depth. Profile: Open flowers are flat. Fragrance: None. Lasting Quality: Flowers normally remain 8 days on the plant.
   Tepals.—Tepal Color: Upon opening, the upper surface is Red-Purple Group 71A with intonations of Red-Purple Group N79C. The lower surface is Purple-Violet N80C on each side of the Yellow-Green 145A central bar. Margins on the lower surface are Red-Purple Group 71 A. After opening, the upper surface is Red-Purple Group 71A. The lower surface is Purple-Violet N80C on each side of the Yellow-Green 145A central bar. Margins on the lower tepal surface are Red-Purple Group 71 A. Quantity: Normally 6 tepals. Size: 60 mm in length by 38 mm wide. Shape: Individual tepal shape is elliptic. The apex is mucronate and the base is acute. Apex Recurvature: Slightly curved. Tepal Cross section: Slightly reflexed. Margins: Entire. Weak undulations of margin observed. Persistence: Tepals drop off cleanly.
   Reproductive organs.—Arrangement: Fully open, the reproductive parts span to a diameter of 30 mm. An inner whorl of anthers remains tightly compact, surrounding the pistils. Pollen: None observed. Anthers: Size: 6 mm in length. Color: Yellow-Green Group 150D. Quantity: On average, 55. Filaments: Color: Yellow-White Group 158D. Length: 5 mm. Pistils: Quantity: On average, 30. Stigmas: Level in location relative to the length of the filaments and the height of the anthers. Styles: Color: Yellow-Green Group 145D. Length: 7 mm.
   Seed head characteristics.—None observed.
Plant:
   Plant form.—Climbing.
   Plant growth.—Moderately vigorous.
   Size.—Seasons growth attains 1 meter in height. Average spread is 60 cm.
   Stems.—Color: Juvenile stems are Yellow-Green Group 144B. Mature stems are Greyed-Orange Group 166A. Internodes: On average, 9 cm between nodes. Length: Normally 40 cm from the base of the plant to the flowering portion of the stem. Diameter: Normally 2 mm. Texture: Mature stems are generally smooth and ribbed.
   Plant foliage.—Leaf characteristics: Deciduous. Arrangement: Trifoliate. Leaf Size: Compound leaves are normally 120 mm (1)×100 mm (w). Leaflets are normally 60 mm (1)×35 mm (w). Abundance: On average 1 compound leaf per 10 cm of stem. Leaf Color: Juvenile upper and lower surfaces are Yellow-Green Group 144A with light anthocyanin the color of Greyed-Red Group 178B at the margins. Mature leaves are Yellow-Green Group 146A on the upper surface, and Yellow-Green Group 146B on the lower surface. Stipules: Absent. Petioles: Size: Normally 40 mm in length by 2 mm diameter. Texture: Smooth. Color: Yellow-Green Group 144B. Petioloule: Size: Normally 10 to 20 mm in length by 1.5 mm diameter. Texture: Smooth. Color: Yellow-Green Group 144B. Leaflet Shape: Generally ovate to cordate. The base is acute or flat. The apex is acute. Margin: Entire. Surface: The upper side is smooth. The lower side is smooth. Thickness: Average. Glossiness: Moderately glossy.
   Disease resistance.—Subject to any disease that normally attacks the species. However the variety is more tolerant to clematis wilt than some clematis.
   Cold hardiness.—The variety is tolerant to USDA Hardiness Zone 6.
   Heat tolerance.—The variety has been found to be suitable for climate conditions found in the American Horticulture Society heat zone 7.

We claim:

1. A new and distinct variety of clematis plant named ‘Evipo043’, substantially as described and illustrated herein, due to its abundant Red-Purple flowers with good keepability, attractive foliage, compact growth, suitability for production from softwood cuttings in pots, which make the variety suitable for distribution in the floral industry.
